(19) United States
(12) Patent Application Publication
(10) Pub. No.: US 2023/0322694 A1
(43) Pub. Date: Oct. 12, 2023

(54) COMPOUND

(71) Applicant: Sumitomo Chemical Co., Ltd, Tokyo (JP)

(72) Inventors: Philip Benzie, Godmanchester (GB); Helen Benjamin, Godmanchester (GB); Andrew Lee, Godmanchester (GB)

(21) Appl. No.: 18/129,363

(22) Filed: Mar. 31, 2023

(30) Foreign Application Priority Data
Mar. 31, 2022 (GB) 2204661.9

(51) Int. Cl. C07D 277/66; C07D 417/10

(52) U.S. Cl. CPC C07D 277/66; C07D 417/10

(57) ABSTRACT

A compound of formula (I):

Ar¹ (I)
Ar¹ is a fused aromatic or heteroaromatic group substituted with at least one group of formula (II):

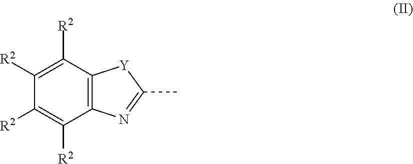

R² in each occurrence is H or a substituent, Y is O, S or NR⁹ wherein R⁹ is H or a substituent; --- is a bond to a ring carbon atom of Ar¹; and, for each group of formula (II), a ring carbon atom of Ar¹ adjacent to a ring carbon atom bound to the group of formula (II) is substituted with a group of formula —XR¹ wherein X is O, S or NR³ wherein R³ is H or C₁₋₁₂ alkyl and R¹ is a photocleavable group. Data may be written to a recording medium containing a compound of formula (I) by exposing it to a wavelength for photolysis of the photocleavable group.

### CROSS REFERENCE TO RELATED APPLICATIONS

This application claims the benefit and priority to United Kingdom Patent Application No. 2204661.9, filed Mar. 31, 2022, the disclosure of which is incorporated herein by reference in its entirety.

### BACKGROUND

Excited state intramolecular proton transfer (ESPIT) dyes are known. For example, Kocher et al, “Aromatic 2-42′-hydroxyphenyl)benzoxazole esters: a novel class of caged photoluminescent dyes”, J. Mater. Chem., 2002, 12, 2620-2626 discloses cleavage of an ester of a non-ionic ‘caged’ photoluminescent dye to produce a photoluminescent dye.

Padalkar et al, “Optical and Structural Properties of ESIPT Inspired HBT-Fluorene Molecular Aggregates and Liquid Crystals”, Phys. Chem. B 2017, 121, 45, 10407-10416 discloses photophysical properties of fluorene-HBT positional isomers in solution and in the solid state.

### SUMMARY

In some embodiments, the present disclosure provides a compound of formula (I):

Ar¹ (I)

wherein:
Ar¹ is a fused aromatic or heteroaromatic group substituted with at least one group of formula (II):

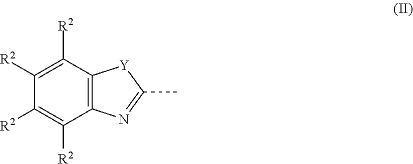

R² in each occurrence is H or a substituent;
Y is O, S or NR⁹ wherein R⁹ is H or a substituent;
--- is a bond to a ring carbon atom of Ar¹;
and, for each group of formula (II), a ring carbon atom of Ar¹ adjacent to a ring carbon atom bound to the group of formula (II) is substituted with a group of formula —XR¹ wherein X is O, S or NR³ wherein R³ is H or C₁₋₁₂alkyl and R¹ is a photocleavable group.

Optionally, Ar¹ is a fused aromatic group.

Optionally, the compound of formula (I) has formula (Ia):

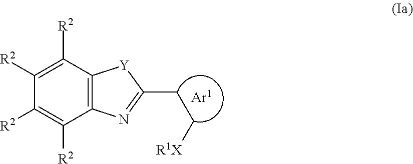

Optionally, Ar¹ of formula (Ia) is selected from formulae (IIIa) and (IIIb):

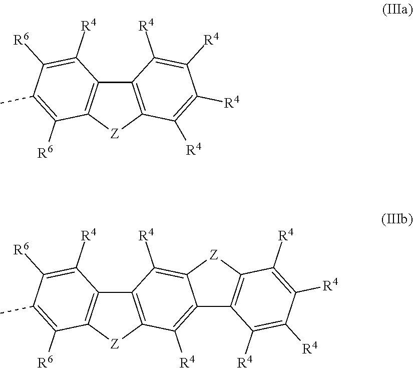

wherein:
Z is O, S, NR¹³, CR⁵₂ or SiR⁵₂, wherein R⁵ in each occurrence is independently a substituent and R¹³ is H or a substituent:
R⁴ in each occurrence is independently H or a substituent; and
each R⁶ is H or a substituent with the proviso that one R⁶ is XR¹.

Optionally, the compound of formula (I) has formula (Ib) or (Ic):

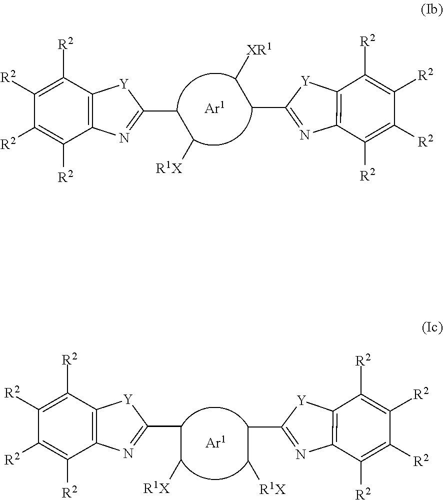

Optionally. Ar¹ of formula (Ib) or (Ic) is selected from formulae (IVa) and (IVb):

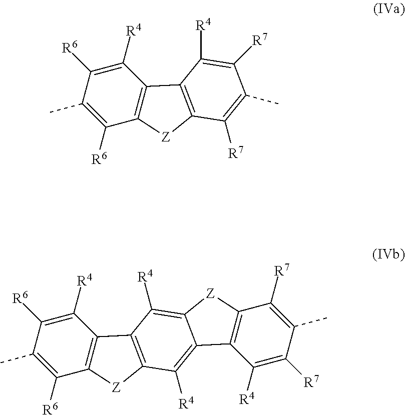

wherein:
Z is O, S, NR¹³, CR⁵₂ or SiR⁵₂ wherein R⁵ in each occurrence is independently a substituent and R¹³ is H or a substituent R⁴ in each occurrence is independently H or a substituent,
each R⁶ is H or a substituent with the proviso that one R⁶ is XR¹, and
each R⁷ is H or a substituent with the proviso that one R⁷ is XR¹.

Optionally. R¹ is a group of formula (V):

—C(═O)R⁸ (V)
wherein R⁸ is a substituent.

According to some embodiments, the present disclosure provides a composition comprising a compound of formula (I) dispersed in a matrix.

According to some embodiments, the present disclosure provides a method of photolysis of a compound of formula (I) comprising irradiating the compound with light of having a wavelength the same as or shorter than an absorption peak of the compound.

According to some embodiments, the present disclosure provides a method of photolysis of a compound of formula (I) comprising irradiating the compound with light of having a wavelength longer than an absorption peak of the compound.

In some embodiments, the present disclosure provides a recording medium comprising a layer comprising a compound or a composition as described herein.

A method of writing data to the recording medium may comprise exposing selected regions of the recording medium to a photolysing beam.

### DESCRIPTION OF DRAWINGS

FIG. 1 is a graph of absorption spectra for Comparative Compound 1, Compound Example 1 and Compound Example 3 (A=absorption; nm=nanometers);

FIGS. 2-4 each show emission spectra for, respectively, Comparative Compound 1, Compound Example 1 and Compound Example 3 and the respective compounds which would be produced by photolysis of these compounds (in FIGS. 2, 3, and 4, x axis is wavelength in nm and y axis is normalized photoluminescent (PL) intensity);

FIG. 5 is computer-modelled 2-photon absorption cross-sections of Compound Examples 1, 2 and 3 (FIG. 5: x axis is wavelength in nm and y axis is absorption (A)); and

FIG. 6 is a photograph of a film containing Compound Example 1 following 1-photon writing with a 420 nm laser, and

FIG. 7 is a photograph of a film containing Compound Example 1 following 2-photon writing with a 532 nm laser.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other systems, not necessarily the system described below. The elements and acts of the various examples described below can be combined to provide further implementations of the technology.

Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that compounds of formula (I) can undergo photolysis to form compounds having higher photoluminescence that compounds of formula (I) and/or having a longer emission and/or absorption wavelength, for example an absorption wavelength of the photolysed compound which is at least 30 nm greater, optionally at least 50 nm greater, than the absorption peak wavelength of the compound of formula (I). The luminance of a photolysed compound as described herein is preferably as measured at an excitation wavelength of at least 500 nm.

The compounds of formula (I) may have an absorption peak in excess of 300 nm. This may allow excitation and photolysis using low energy (long wavelength) light sources which may reduce or avoid damage to the material as compared to use of a higher energy light source.

The Stokes shift of a photolysed compound of formula (I) is preferably at least 50 nm, more preferably at least 80 nm.

The compound of formula (I) is a fused aromatic or heteroaromatic group Ar¹ which is substituted with one or more groups of formula (II):

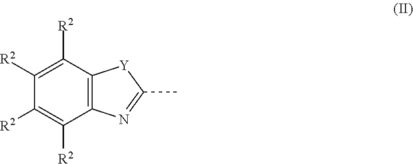

Y is O, S or NR⁹ wherein R⁹ is H or a substituent. Preferably, R⁹ is selected from H and C₁₋₁₂alkyl. More preferably R⁹ is H.
R² in each occurrence is independently H or a substituent and --- is a bond to a ring carbon atom of Ar¹.
R² may be H; F; aryl or heteroaryl, preferably phenyl, which is unsubstituted or substituted with one or more substituents; and linear, branched or cyclic C₁₋₂₀ alkyl wherein one or more non-adjacent, non-terminal C atoms of the alkyl may be replaced with O and one or more H atoms of the alkyl may be replaced with F.

Substituents of an aryl or heteroaryl group R² are preferably selected from F and linear, branched or cyclic C₁₋₂₀alkyl wherein one or more non-adjacent, non-terminal C atoms of the alkyl may be replaced with O and one or more H atoms of the alkyl may be replaced with F.

By “non-terminal C atom” of an alkyl chain is meant a C atom other than the C atom of the methyl group of a linear alkyl chain or the C atoms of the methyl groups of a branched alkyl chain.

For each group of formula (II), Ar¹ is substituted with a group —XR¹ at a ring carbon atom adjacent to the ring carbon atom which is bound to the group of formula (II). X is O, S or NR³ wherein R³ is H or C₁₋₁₂ alkyl and R¹ is a photocleavable group.

The one or more groups —XR¹ may be the only substituents of Ar¹, or Ar¹ may be substituted with one or more further substituents.

Preferably, Ar¹ is a fused aromatic or heteroaromatic group wherein the fused group comprises at least 2 benzene rings, more preferably a C₁₀₋₁₀ aromatic or heteroaromatic group. Ar¹ is preferably fluorene or indenofluorene.

Preferably, Ar¹ is bound to 1, 2 or 3 groups of formula (I), more preferably to 1 or 2 groups of formula (II).

In the case where Ar¹ is substituted with only one group of formula (II), the compound of formula (I) has formula (Ia):

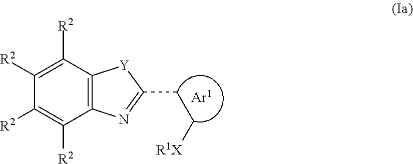

Ar¹ of formula (Ia) is an aryl or heteroaryl group, more preferably an aryl group. Particularly preferred aryl groups Ar¹ are formulae (IIIa) and (IIIb):

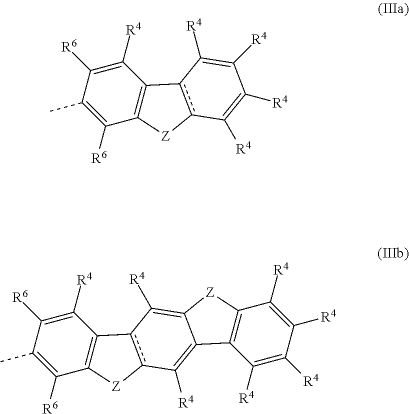

R⁴ in each occurrence is independently H or a substituent;
Z is O, S, NR¹³, CR⁵₂ or SiR⁵₂ wherein R⁵ in each occurrence is independently a substituent and R; is H or a substituent; and
each R⁴ is H or a substituent with the proviso that one R⁶ is XR¹.

Each R⁴ may be H or a substituent as described with respect to R². Preferably, each R⁴ is H.

Preferably, each Z is CR⁵₂.

Each R⁵ is preferably selected from aryl or heteroaryl, preferably phenyl, which is unsubstituted or substituted with one or more substituents; and linear, branched or cyclic C₁₋₂₀ alkyl wherein one or more non-adjacent, non-terminal C atoms of the alkyl may be replaced with 0 and one or more H atoms of the alkyl may be replaced with F.

Substituents of an aryl or heteroaryl group R⁵ are preferably selected from F and linear, branched or cyclic C₁₋₂₀ alkyl wherein one or more non-adjacent, non-terminal C atoms of the alkyl may be replaced with 0 and one or more H atoms of the alkyl may be replaced with F.

R¹³ is preferably selected from H; C₁₋₂₀ alkyl; and unsubstituted or substituted aryl, e.g. phenyl. Exemplary substituents of the aryl group are F, CN, NO₂, and C₁₋₁₂ alkyl.

Preferably, one R⁶ is XR¹ and the other R⁶ is selected from H or C₁₋₁₂ alkyl, preferably H.

In the case where Ar¹ is substituted with two groups of formula (II), the compound of formula (I) has formula (Ib) or (Ic):

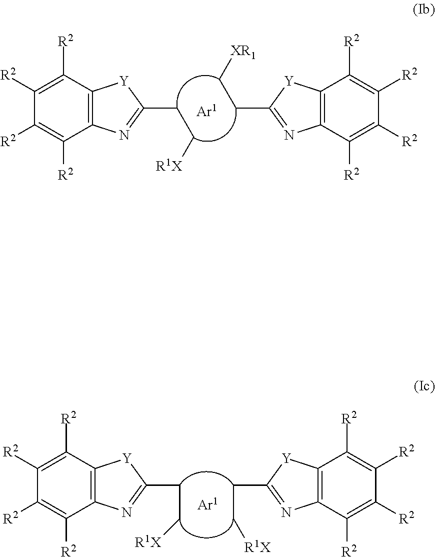

Ar¹ of formula (Ib) or (Ic) is an arylene or heteroarylene group, more preferably an arylene group. Particularly preferred arylene groups Ar¹ are formulae (IVa) and (IVb):

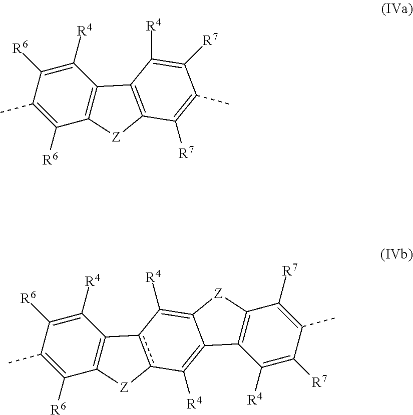

Z, R⁴, and R⁶ are as described above.

Each R⁷ is H or a substituent with the proviso that one R⁷ is XR¹.

Preferably, one R⁷ is XR¹ and the other R⁷ is selected from H or C₁₋₁₂ alkyl, preferably H.

Preferably, R¹ is a group of formula (V):

—C(═O)R⁸ (V)
wherein R⁸ is a substituent, preferably a C₁₋₂₀alkyl or an aromatic or heteroaromatic group which may be unsubstituted or substituted with one or more substituents.

Substituents of an aryl or heteroaryl group R⁸ are preferably selected from F and linear, branched or cyclic C₁₋₂₀alkyl wherein one or more non-adjacent, non-terminal C atoms of the alkyl may be replaced with O and one or more H atoms of the alkyl may be replaced with F.

Preferably X is O.

Upon photolysis, the X—R¹ bond is cleaved and the photolysed compound the, or each, X—R¹ group becomes an X—H group. Preferably, R¹ is selected from the group consisting of R¹⁰C(═O)—, R¹⁰C(═O)CH₂—, R¹⁰OC(═O)—, R¹¹ and

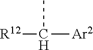

wherein:
R¹⁰ is selected from the group consisting of C₁₋₂₀ alkyl and aryl or heteroaryl which is unsubstituted or substituted with one or more substituents;
R¹¹ is an aryl or heteroaryl which is unsubstituted or substituted with one or more substituents;
R¹² is C₁₋₂₀alkyl, or aryl or heteroaryl which is unsubstituted or substituted with one or more substituents; and
Ar² is an unsubstituted or substituted phenyl.

An aryl or heteroaryl group R¹⁰, R¹¹ or R¹² is preferably phenyl.

Substituents of an aryl or heteroaryl group R¹⁰, R¹¹ or R¹² or a phenyl group Ar2 are preferably selected from F, Cl, NO₂, CN, C₁₋₁₂ alkyl and C₁₋₁₂alkoxy, preferably NO₂ and C₁₋₁₂alkoxy.

Exemplary groups R¹ include:

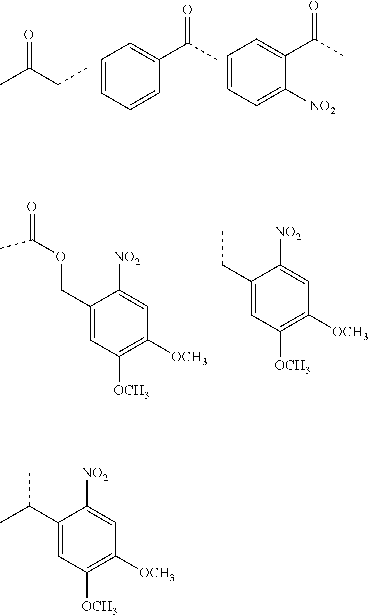

Exemplary compounds of formula (I) include:

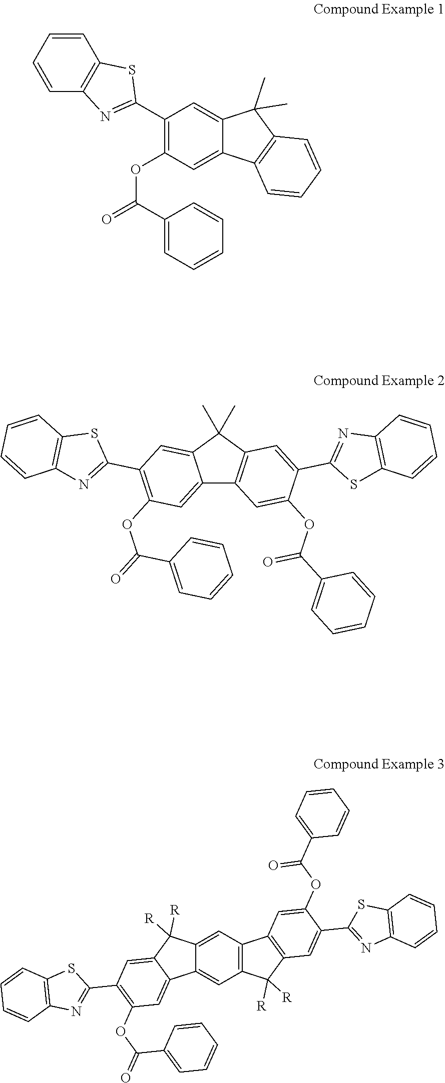

The material produced upon photolysis of a compound of formula (I) preferably has higher photoluminescent quantum yield (PLQY) at 20° C., optionally higher by at least a factor of 2, a factor of 5 or a factor of 10, as compared to PLQY of the compound of formula (I).

Optionally, the material produced upon photolysis has a peak emission wavelength greater than 400 nm, more preferably greater than 450 nm or 500 nm.

The compound of formula (I) may be used in any application requiring a material capable of changing its emission wavelength and/or increasing in luminance upon irradiation.

A composition may contain a compound of formula (I) and a matrix. The matrix may be, for example, a polymer.

The compound of formula (I) may be used to write data to a recording medium. The recording medium may take any form configured to be written to by a writing apparatus, for example a disc. The disc may contain a single writeable layer or may contain two or more writeable layers. The disc may be single sided or double sided.

In some embodiments, a composition comprising a compound of formula (I) is irradiated in selected regions with light having a write wavelength to cause photolysis of the compound.

In some embodiments, the composition is irradiated with a light from a light source having a write wavelength that is the same as or shorter than an absorption peak of the compound of formula (I). Preferably, the light source is a laser.

In some embodiments, data is written by a 2-photon absorption method. Two-photon absorption is described in “Three-Dimensional Microfabrication Using Two-Photon Polymerisation”, Ed. Tommaso Baldacchini, Elsevier 2016, the contents of which are incorporated herein by reference.

In 2-photon absorption, the compound of formula (I) absorbs two photons of the write wavelength λ₁ causing excitation from a ground state, via a virtual state, to an excited state. The energy 2hc/λ₁, absorbed by the absorbing material is at least the same as or higher than that of the ground state—excited state energy gap of the material. 2-photon absorption allows for the wavelength λ₁ to be longer than an absorption peak of the compound of formula (I). Optionally. λ₁ is at least 500 nm, optionally in the range of about 500-1000 nm.

Peak power of the write beam to achieve 2-photon absorption is preferably in the range of 1-100 KW.

Due to its non-linear nature, 2-photon absorption is particularly advantageous for writing to a specified depth of a recording medium, e.g. for a 3D recording or recording to a recording medium having more than one writable layer.

Reading apparatus configured to read data recorded on the recording medium may comprise a light source configured to emit an excitation beam onto the recording medium wherein the excitation beam has a wavelength at which the photolysed compound of formula (I) luminesces.

The written recording medium may be read by illuminating the recording medium and reading emission from the written recording medium. In some embodiments, the written recording medium is read by stimulated emission depletion (STED) in which the excitation beam is surrounded by a deactivation beam such that only organic luminescent material irradiated by the excitation beam in a focal area emits light. The skilled person will understand how the focal area may be adjusted by altering the properties of the pupil plane of an objective lens. STED is described in more detail in, for example, Gu et al, “Nanomaterials for optical data storage”, Nature Reviews Materials, Vol 1, p. 1-14, December 2016, the contents of which are incorporated herein by reference.

### EXAMPLES

### Example 1

### 9,9-dimethyl-9H-fluorene-2-carbaldehyde

n-BuLi (6.77 ml, 2.36 M) was added slowly to a stirred solution of 2-bromo-9,9-dimethyl-9H-fluorene (4.00 g, 14.6 mmol) in THF (100 ml, dry) at −78° C. During addition the solution turned yellow, and towards the end of the addition a white precipitate formed. After stirring at the same temperature for 1 hour, the yellowish solution was warmed up to −30° C., and then cooled back to −78° C. Anhydrous DMF was then added slowly, and the solution was stirred at −78° C. for 2 hours before being warmed up to room temperature. The reaction was quenched by addition of 1 M HCl (100 mL), and extracted with ethyl acetate (3×100 mL). The combined organic layers were dried over Mg₂SO₄. After removal the solvent under vacuum, the residue was purified by a column chromatography (6-50% EtOAc in heptane). Product was obtained as a pale yellow oil (2.77 g)

### 2-(9,9-dimethyl-9H-fluoren-2-yl)benzo[d]thiazole

2-aminothiophenol (1.26 g, 10.03 mmol) and 9,9-dimethyl-9H-fluorene-2-carbaldehyde (2.77 g, 12.4 mmol) were combined in DMSO (20 ml). The mixture was then heated for 6 hours at 60° C. before being cooled to room temperature. Water (50 ml) was added and the mixture was extracted with EtOAc (3×50 ml). The combined organic layers were then washed with brine, dried with MgSO₄, filtered and the solvent removed in vacuo. The material was purified by column chromatography (heptane:EtOAc) to give a white solid (2.70 g, 64%)

### 2-(benzo[d]thiazol-2-yl)-9,9-dimethyl-9H-fluoren-3-yl acetate

9,9-dimethyl-9H-fluoren-2-yl)-1,3-benzothiazole (2.70 g, 8.24 mmol), Pd(OAc)₂ (92 mg, 0.41 mmol), and (diacetoxyiodo)benzene (2.65 g, 8.24 mmol) were combined in glacial acetic acid (20 ml) and heated to reflux for 5 hours under air before being cooled to room temperature. Water (100 ml) was added, and the mixture was extracted with EtOAc (3×100 ml). The organic fractions were combined, dried over MgSO₄, filtered and the solvent removed in vacuo. The residue was purified by column chromatography (heptane:EtOAc, stepwise—18% then 33%). Unreacted starting material eluted first, followed by the product as a red tinted oily solid. The material was used for the next step without further purification (1.38 g, 44%); δ [600 MHz, CDCl₃, ppm] 1.58 (s, 6H), 2.54 (s, 3H) 7.34-7.43 (m, 3H), 7.47 (d, J=6.7 Hz, 1H), 7.52 (t, J=7.8 Hz, 1H), 7.56 (s, 1H), 7.72 (d, J=7.0 Hz, 1H), 7.9375 (d, J=7.8 Hz, 1H), 8.12 (d, J=8.2 Hz, 1H), 8.4 (s, 1H)

### 2-(benzo[d]thiazol-2-yl)-9,9-dimethyl-9H-fluoren-3-ol

K₂CO₃ (1.96 g, 14.2 mmol) was added to a stirred solution of 2-(1,3-benzothiazol-2-yl)-9,9-dimethyl-9H-fluoren-3-yl acetate (1.38 g, 3.57 mmol) in methanol (50 ml) at room temperature. Immediately, the solution turned yellow and began to fluoresce. The mixture was stirred at room temperature for 5 hours before being filtered through paper to remove any solid. The solvent was then removed in vacuo. The yellow residue was then resuspended in EtOAc (100 ml), and water (100 ml) was added. The aqueous layer was then neutralised to pH 7 by addition of dilute HCl solution (monitored with pH paper). Once neutral, the mixture was shaken in a separating funnel, and separated. The aq portion was washed with further EtOAc (100 ml), and the organic phases were combined, dried over MgSO₄ and solvent removed. The mixture was then purified by column chromatography (50/50 heptane:DCM). The product was obtained as a bright yellow solid (1.08 g, 89%); δ [600 MHz, DMSO-d₆, ppm] 2.50 (s, 6H), 7.36-7.42 (m, 2H), 7.448 (t, J=7.2 Hz, 1H), 7.492 (s, 1H), 7.549 (t, J=7.2 Hz, 1H), 7.587 (d, J=6.0 Hz, 1H), 7.849 (d, J=6.2 Hz, 1H), 8.082 (d, J=8.0 Hz, 1H), 8.146 (d, J=8.0 Hz, 1H), 8.26 (s, 1H), 11.79 (s, 1H)

### 2-(benzo[d]thiazol-2-yl)-9,9-dimethyl-9H-fluoren-3-yl benzoate

2-(benzo[d]thiazol-2-yl)-9,9-dimethyl-9H-fluoren-3-ol (1.08 g, 3.14 mmol)_was dissolved in CHCl₃ (100 ml) and pyridine (0.77 ml, 9.57 mmol) added. Benzoyl chloride (0.578 ml, 4.99 mmol) was then added dropwise at room temperature under an atmosphere of N₂, and the mixture was heated to reflux overnight before being cooled to room temperature. During this time, the yellow fluorescent solution turned almost completely colourless. Water was added, and the layers separated. The aqueous layer was extracted further with EtOAc (2×50 ml), before the layers were combined, dried over MgSO₄, filtered and solvent removed. The residue was purified by column chromatography (Toluene 100%). The material was triturated with heptane (3×10 ml). The product was obtained as a white solid (1.16 g, 83%); δ [600 MHz, CDCl₃, ppm]1.62 (s, 6H), 7.32 (td, J=7.6, 1.0 Hz, 1H), 7.36 (td, J=7.3, 1.1 Hz, 1H), 7.40 (td, J=7.2, 1.2 Hz, 1H), 7.45 (td, J=7.1, 1.1 Hz, 1H), 7.49 (d, J=7.4 Hz, 1H), 7.58-7.62 (m, 2H), 7.63 (s, 1H), 7.70-7.74 (m, 2H), 7.81 (d, J=7.8 Hz, 1H), 7.98 (d, J=8.1 Hz, 1H), 8.35-8.38 (m, 2H), 8.49 (s, 1H)

### Example 2

Example 2 was prepared according to the following scheme:

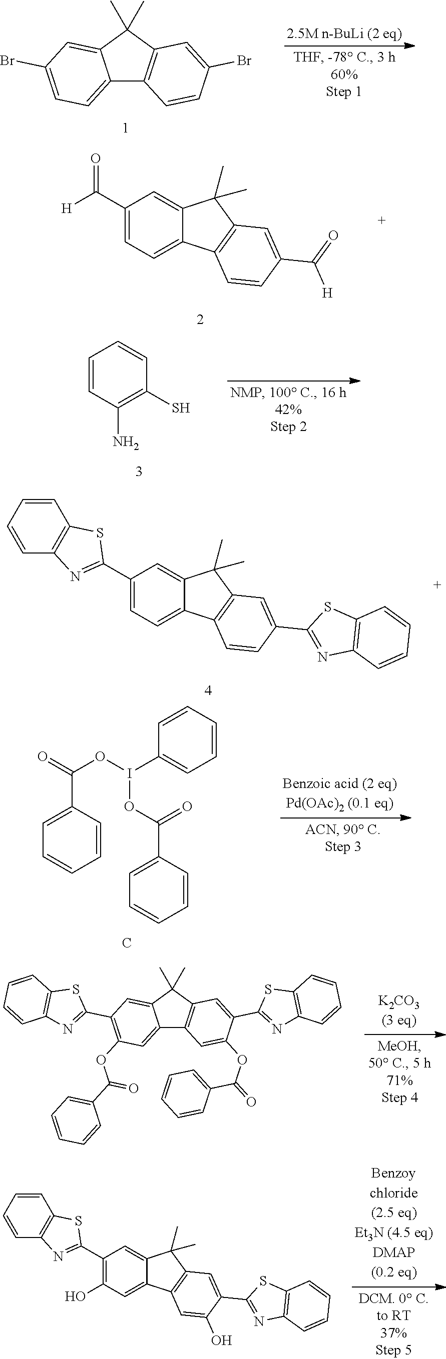

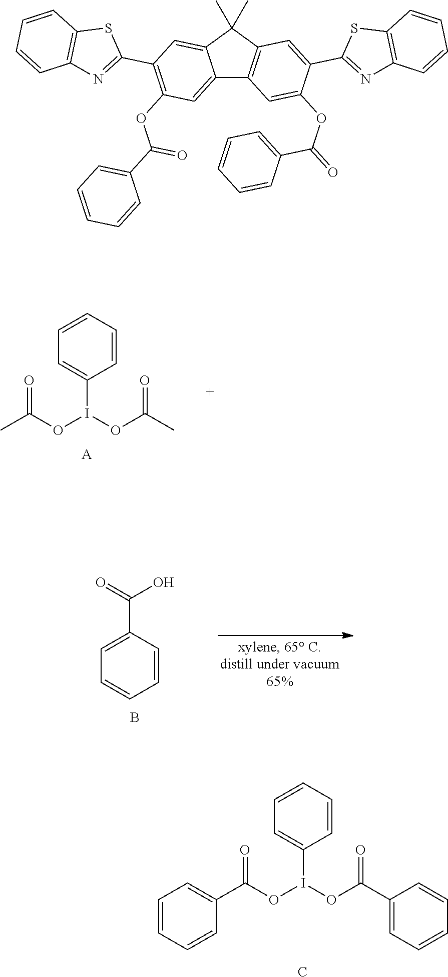

### 9,9-dimethyl-9H-fluorene-2,7-dicarbaldehyde

n-BuLi (98.2 ml, 2.5 M) was added slowly to a stirred solution of 2,7-dibromo-9,9-dimethyl-9H-fluorene (40 g, 114 mmol) in THF (800 ml, dry) at −78° C. The reaction was maintained at −78° C. for 1.5 hours before DMF (250 ml) was added dropwise. The reaction mixture was stirred at −78° C. for 1.5 hours before being quenched by dropwise addition of water (300 ml). The mixture was diluted with EtOAc, and the organic layer was separated and concentrated to give 60 g of crude material. The crude product was dissolved in ACN (500 mL) and then stirred at 25° C. for an hour. The solid formed was filtered and resuspended in ACN. The solid was collected by filtration to give the product (17 g, 60%); δ [ppm] 1.60 (s, 6H), 7.93-7.99 (m, 4H), 8.04 (s, 2H), 10.12 (s, 2H).

### 2,2′-(9,9-dimethyl-9H-fluorene-2,7-diyl)bis(benzo[d]thiazole)

9,9-dimethyl-9H-fluorene-2,7-dicarbaldehyde (18 g, 71.9 mmol) was dissolved in NMP (400 ml) and 2-aminothiophenol (26.9 g, 215 mmol) was added. The reaction mixture was purged with oxygen for 30 minutes before being heated to 100° C. for 16 hours. The reaction mixture was cooled to room temperature and quenched by addition of water (1 L) and extracted with EtOAc. The organic phase was concentrated to give a solid. The solid was triturated with acetonitrile and filtered to give 2,2′-(9,9-dimethyl-9H-fluorene-2,7-diyl)bis(benzo[d]thiazole) (14 g, 42%); δ [ppm] 1.69 (s, 6), 7.43 (t, J=7.20 Hz, 2H), 7.54 (t, J=7.20 Hz, 2H), 7.89 (d, J=8.00 Hz, 2H), 7.95 (d, J=7.60 Hz, 2H), 8.11 (d, J=8.00 Hz, 2H), 8.14 (d, J=8.40 Hz, 2H), 8.29 (s, 2H).

### 2,7-bis(benzo[d]thiazol-2-yl)-9,9-dimethyl-9H-fluorene-3,6-diyl dibenzoate

A solution of 2,2′-(9,9-dimethyl-9H-fluorene-2,7-diyl)bis(benzo[d]thiazole) (5 g, 10.8 mmol) in ACN (450 ml) was degassed for 30 min. Benzoic acid (2.63 g, 21.6 mmol) and PhI(O₂CPh)₂ (9.64 g, 21.6 mmol) were added to the reaction mixture and degassed with nitrogen for an hour. Palladium acetate (242 mg, 1.08 mmol) was added and the reaction mixture was heated to 90° C. and stirred for 4 days before being cooled to room temperature. The reaction mixture was diluted with DCM (500 mL) and washed with sodium bicarbonate solution (300 mL). The organic phase was separated and concentrated to give the crude product. In order to purify, the compound was transformed to the alcohol.

### 2,7-bis(benzo[d]thiazol-2-yl)-9,9-dimethyl-9H-fluorene-3,6-diol

Crude 2,7-bis(benzo[d]thiazol-2-yl)-9,9-dimethyl-9H-fluorene-3,6-diyl dibenzoate (1.1 g, 1.56 mmol) and potassium carbonate (646 mg, 4.68 mmol) were taken in methanol (40 mL). The reaction was heated to 50° C. for 5 hours before being cooled, diluted with water and neutralized with dilute HCL. The mixture was extracted with DCM (×3) and combined organics were washed with water, saturated brine solution and dried over anhydrous sodium sulphate. The organic phase was concentrated to give the crude product, which was dissolved in methanol, precipitated into water and stirred for an hour. The solid was collected via filtration, dissolved in EtOAc, washed with water and dried over sodium sulphate. The solvent was removed in vacuo to give the product (550 mg, 71%): δ [400 MHz, DMSO-d₆, ppm] 1.59 (s, 6H), 7.45-7.45 (m, 4H), 7.57 (t, J=8.40 Hz, 2H), 8.12 (d, J=8.00 Hz, 2H), 8.17 (d, J=8.00 Hz, 2H), 8.34 (s, 2H), 11.80 (s, 2H).

### 2,7-bis(benzo[d]thiazol-2-yl)-9,9-dimethyl-9H-fluorene-3,6-diyl dibenzoate

2,7-bis(benzo[d]thiazol-2-yl)-9,9-dimethyl-9H-fluorene-3,6-diol (0.45 g, 0.913 mmol), Triethylamine (0.57 mL, 4.10 mmol) and DMAP (22.2 mg, 182 μmol) were combined in DCM (15 mL) and cooled to 0° C. A solution of benzoyl chloride (320 mg, 2.28 mmol) in DCM (1 mL) was slowly added at 0° C. The mixture was allowed to warm to room temperature and stirred for 16 h. The reaction mixture was diluted with water (50 mL) and extracted with dichloromethane (50 mL). The organic phase was washed with water and saturated brine solution, dried over anhydrous sodium sulphate and the solvent removed to give the crude product (0.7 g). The crude product was recrystallised from a mixture of ACN/Toluene, to give the product (0.58 g, 64%); δ [400 MHz, CDCl₃, ppm]1.60 (s, 6H), 7.38 (t, J=7.60 Hz, 2H), 7.62 (t, J=7.60 Hz, 4H), 7.69 (s, 2H), 7.73-7.73 (m, 2H), 7.86 (d, J=7.60 Hz, 2H), 8.04 (d, J=8.00 Hz, 2H), 8.37-8.38 (m, 4H), 8.59 (s, 2H).

### Example 3

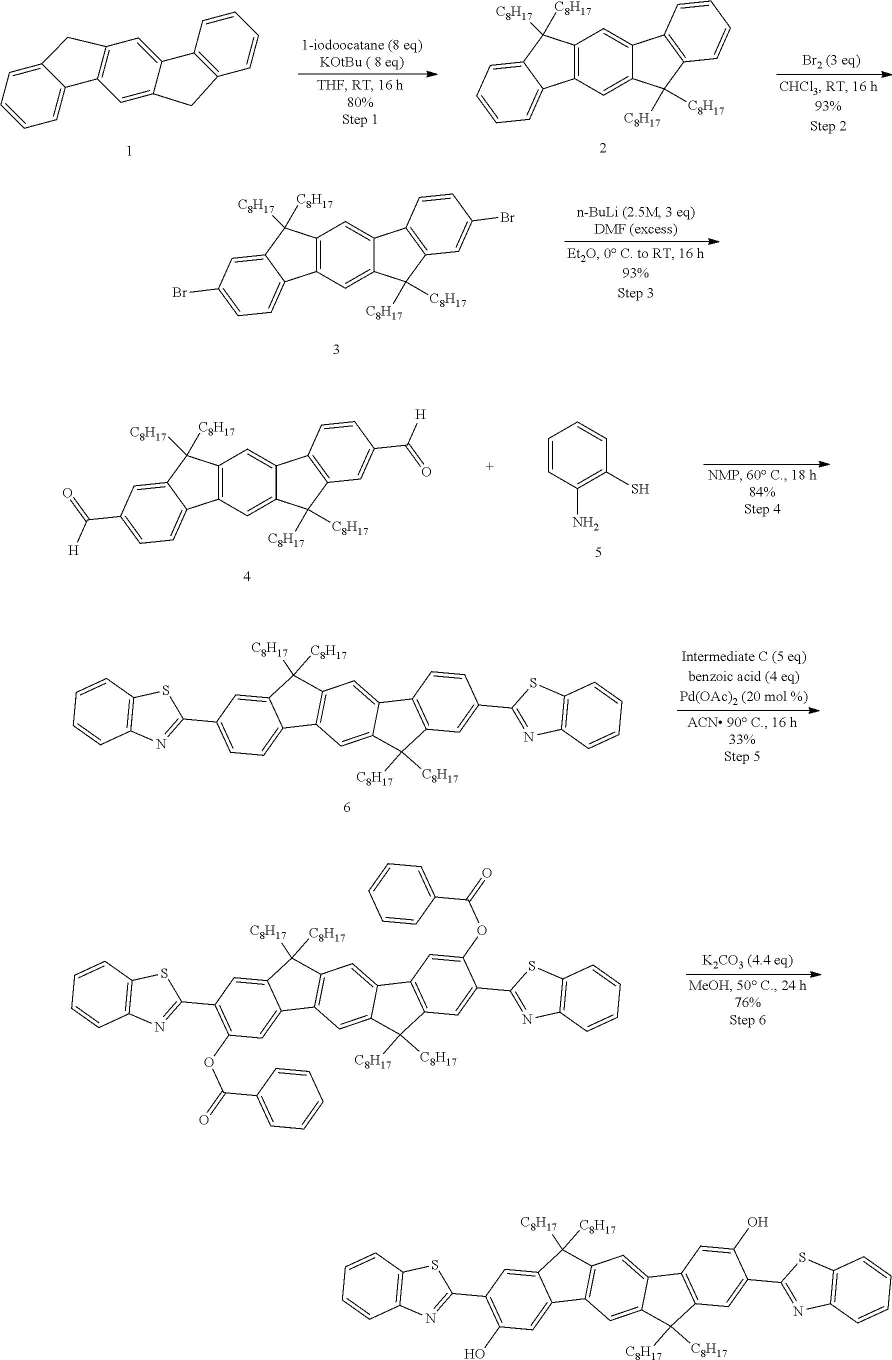

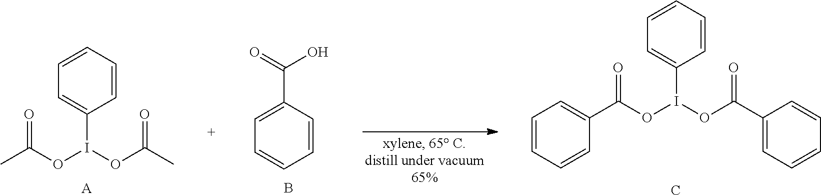

### 6,6,12,12-tetraoctyl-6,12-dihydroindeno[1,2-b]fluorene

6H,12H-indeno[1,2-b]fluorene (10 g, 0.039 mol) was dissolved in THF (400 mL) and cooled to 0° C. Potassium tert-butoxide (35.2 g, 0.314 mol) was added to the reaction mixture at 0° C., and the mixture was allowed to warm to room temperature over 2 hours. The mixture was then cooled to 0° C. and 1-iodooctane (75.4 g, 0.314 mol) was added slowly and the mixture was stirred at room temperature for 16 hours. The reaction mixture was diluted with ethyl acetate and passed a plug of celite. The filtrate was concentrated to give the crude product, which was purified by SiO₂ column chromatography (eluent: hexane) to give the product (24 g @ 97.51% purity). The product was further purified by crystallization in a mixture of 1:5 toluene and acetonitrile, (22 g, 80%): δ [400 MHz, CDCl₃, ppm] 0.669 (bs, 8H) 0.81 (t, J=7.20 Hz, 12H), 1.06-1.22 (m, 40H), 2.03 (q, J=4.80 Hz, 8H), 7.31 (t, J=6.80 Hz, 2H), 7.34-7.35 (m, 6H), 7.76 (d, J=8.40 Hz, 2H).

### 2,8-dibromo-6,6,12,12-tetraoctyl-6,12-dihydroindeno[1,2-b]fluorene

6,6,12,12-tetraoctyl-6,12-dihydroindeno[1,2-b]fluorene (10 g, 14.2 mmol) was dissolved in chloroform (130 mL) and cooled to 0° C. A solution of bromine (6.80 g, 42.6 mmol) in chloroform (20 mL) was added dropwise. The reaction mixture was gradually warmed to room temperature (25° C.) and stirred at room temperature (25° C.) for 16 hours. The reaction mixture was then cooled to 0° C. and quenched with ice water, followed by dilution with DCM. The organic layer was separated and washed with water, saturated brine solution, dried over anhydrous sodium sulphate and concentrated to give the crude product. The material was purified by SiO₂ column chromatography (Eluent: hexane) to give the product (12 g, 98%) δ [400 MHz, CDCl₃, ppm] 0.639 (bs, 8H), 0.81 (t, J=7.20 Hz, 12H), 1.06-1.22 (m, 40H), 2.00 (t, J=8.40 Hz, 8H), 7.49 (d, J=6.80 Hz, 4H), 7.57 (s, 2H), 7.62 (d, J=7.20 Hz, 2H).

### 6,6,12,12-tetraoctyl-6,12-dihydroindeno[1,2-b]fluorene-2,8-dicarbaldehyde

2,8-dibromo-6,6,12,12-tetraoctyl-6,12-dihydroindeno[1,2-b]fluorene (11 g, 12.7 mmol) was dissolved in diethyl ether (220 mL) and cooled to 0° C. n-BuLi (2.43 g, 38.3 mmol) was added slowly at 0° C. and the reaction mixture was stirred at 0° C. for 2 hours. DMF (27 mL) was added dropwise at 0° C., and the mixture was allowed to warm to room temperature and stir for 16 hours. The reaction mixture was then cooled to 0° C. and diluted with ethyl acetate. The layers were separated and the organic layer was washed with water, saturated brine solution and dried over anhydrous sodium sulphate. The solvent was removed in vacuo and the crude product was purified by trituration in hexane (9 g, 93%); δ [400 MHz, CDCl₃,_ppm] 0.63 (t, J=7.20 Hz, 8H), 0.79 (t, J=7.20 Hz, 12H), 1.05-1.28 (m, 40H), 2.06-2.15 (m, 8H), 7.76 (s, 2H), 7.91-7.93 (m, 6H), 10.10 (s, 2H).

### 2,2′-(6,6,12,12-tetraoctyl-6,12-dihydroindeno[1,2-b]fluorene-2,8-diyl)bis(benzo[d]thiazole)

6,6,12,12-tetraoctyl-6,12-dihydroindeno[1,2-b]fluorene-2,8-dicarbaldehyde (2.8 g, 3.68 mmol) was dissolved in NMP (60 mL) and 2-aminothiophenol (1.38 g, 10.8 mmol) was added. The reaction mixture was purged with oxygen for 1 hour before being heated to 60° C. for 18 hours. The reaction mixture was cooled to room temperature and quenched by addition of water (1 L) and extracted with EtOAc. The organic phase was washed with water, saturated brine solution, dried over anhydrous sodium sulphate and then concentrated. The residue was purified by SiO₂ column chromatography (Eluent: 30% DCM in hexane) to give the product (3 g, 84%); δ [400 MHz, CDCl₃, ppm] 0.68-0.74 (m, 8H), 0.77 (t, J=7.20 Hz, 12H), 1.06-1.28 (m, 40H), 2.12-2.20 (m, 8H), 7.42 (t, J=8.00 Hz, 2H), 7.54 (t, J=8.40 Hz, 2H), 7.73 (s, 2H), 7.89 (d, J=8.00 Hz, 2H), 7.95 (d, J=7.60 Hz, 2H), 8.10-8.10 (m, 6H).

### 2,8-bis(benzo[d]thiazol-2-yl)-6,6,12,12-tetraoctyl-6,12-dihydroindeno[1,2-b]fluorene-3,9-diyl dibenzoate

A solution of 2,2′-(6,6,12,12-tetraoctyl-6,12-dihydroindeno[1,2-b]fluorene-2,8-diyl)bis(benzo[d]thiazole) (3 g, 3.09 mmol) in ACN (250 ml) was degassed with nitrogen for 30 min. Benzoic acid (1.5 g, 12.3 mmol) and PhI(O₂CPh)₂ (11 g, 24.7 mmol) were added to the reaction mixture and degassed with nitrogen for an hour. Palladium acetate (138 mg, 0.62 mmol) was added and the reaction mixture was heated to 90° C. for 16 hours. LCMS analysis showed 53% desired product, as a result additional PhI(O₂CPh)₂ (4 g, 8.96 mmol) was added and the reaction mixture was heated to 90° C. for 16 hours before being cooled to room temperature. The reaction mixture was diluted with dichloromethane (200 mL) and washed with 10% sodium bicarbonate solution (300 mL). The layers were separated and the organic layer was washed with water, saturated brine solution, dried over sodium sulphate and concentrated under reduced pressure. The residue was purified by SiO₂ column chromatography (eluent: 30% DCM in hexane), followed by several recrystallisations from a mixture of hot acetonitrile and toluene to give the product (1.25 g, 33%); 6 [400 MHz, CDCl₃, ppm] 0.79 (t, J=6.80 Hz, 20H), 1.11-1.13 (m, 40H), 2.06-2.07 (m, 4H), 2.17-2.19 (m, 4H), 7.36 (t, J=8.40 Hz, 2H), 7.48 (t, J=8.00 Hz, 2H), 7.63-7.63 (m, 6H), 7.75-7.75 (m, 4H), 7.85 (d, J=7.20 Hz, 2H), 8.02 (d, J=8.00 Hz, 2H), 8.42 (d, J=7.20 Hz, 4H), 8.44 (s, 2H).

### 2,8-bis(benzo[d]thiazol-2-yl)-6,6,12,12-tetraoctyl-6,12-dihydroindeno[1,2-b]fluorene-3,9-diol

2,8-bis(benzo[d]thiazol-2-yl)-6,6,12,12-tetraoctyl-6,12-dihydroindeno[1,2-b]fluorene-3,9-diyl dibenzoate (0.2 g, 0.165 mmol) and potassium carbonate (50.1 mg, 0.363 mmol) were combined in methanol (5 mL). The reaction was heated to 50° C. for 5 hours before further potassium carbonate (50.1 mg, 0.363 mmol) was added. Heating continued for an additional 8 hours before the mixture was cooled, diluted with dichloromethane, and washed with water followed by saturated brine solution, dried over anhydrous sodium sulphate and concentrated under reduced pressure. The residue was purified by recrystallisation from a mixture of hot acetonitrile and toluene to give the product (0.125 g, 76%); δ [400 MHz, CDCl₃, ppm] 0.78 (t, J=7.20 Hz, 20H), 1.09-1.13 (m, 40H), 2.08 (t, J=8.00 Hz, 8H), 7.44-7.45 (m, 2H), 7.49 (s, 2H), 7.54-7.54 (m, 2H), 7.61 (s, 2H), 7.67 (s, 2H), 7.95 (d, J=7.20 Hz, 2H), 8.01 (d, J=7.60 Hz, 2H), 12.73 (bs, 2H).

Measurements

Absorption as described herein was measured on dilute solutions of the materials in toluene, or thin films which comprised 5% of the example material, and 95% PMMA by weight. Spectra were measured using a Cary 5000 UV-VIS-NIR Spectrometer.

Luminescence as described herein was as measured on thin films of the material, which comprised of 10% of the example material, and 90% PMMA. In the case of the ‘cleaved’ compounds the emission spectra was recorded of the analogue without the photocleavable group (ie XR¹═OH). This was compared to the emission obtained from material that had been photolyzed and was found to be a good match. Luminescence was measured using a C9920-02 PL Absolute Quantum Yield Measurement System supplied by Hamamatsu, consisting of:
150W Xenon lamp source with fibre optic.
Excitation light guide.
Monochromator.
Integrating sphere.
PMA-12 detector.

Absorption

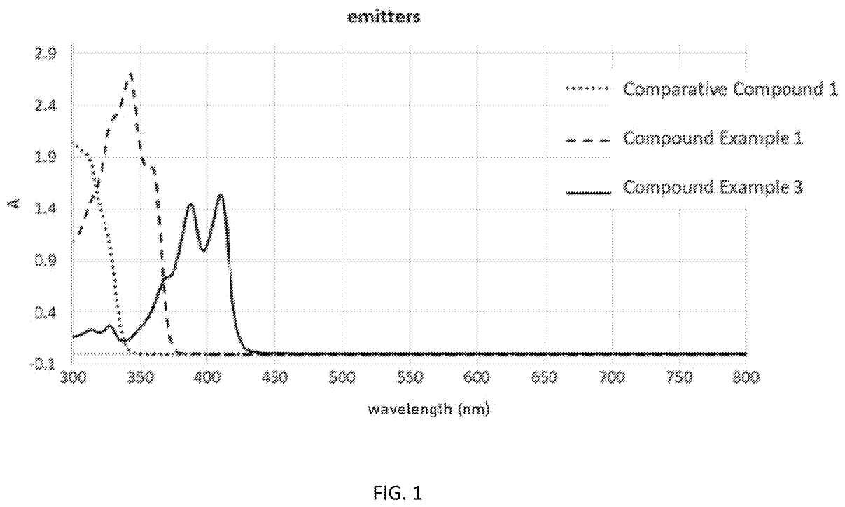

With reference to FIG. 1, Compound Examples 1 and 3 (illustrated above) absorb at a longer wavelength than Comparative Compound 1.

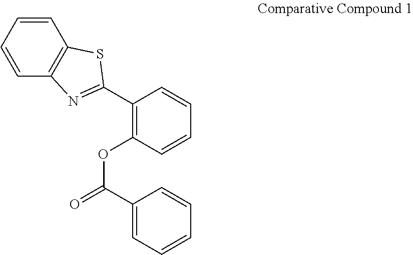 [SMILES: O=C(Oc1ccccc1-c1nc2ccccc2s1)c1ccccc1]

Emission

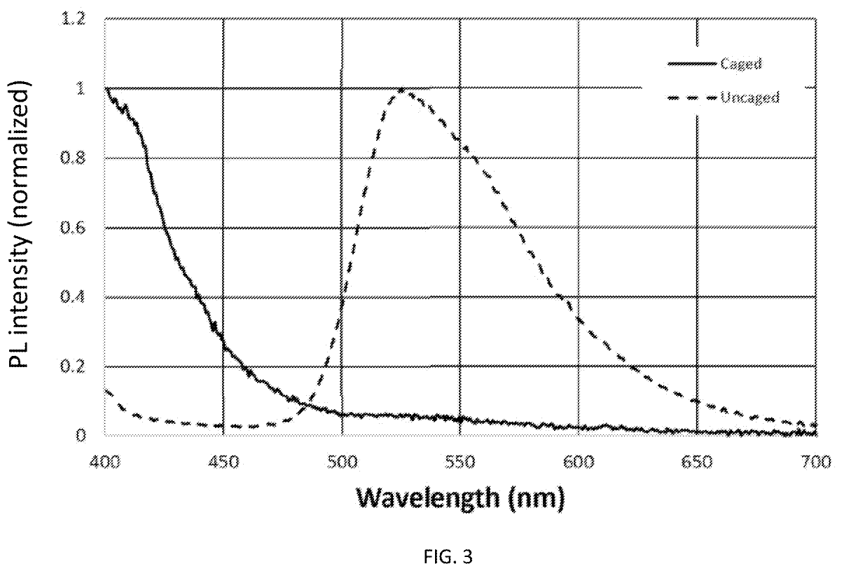

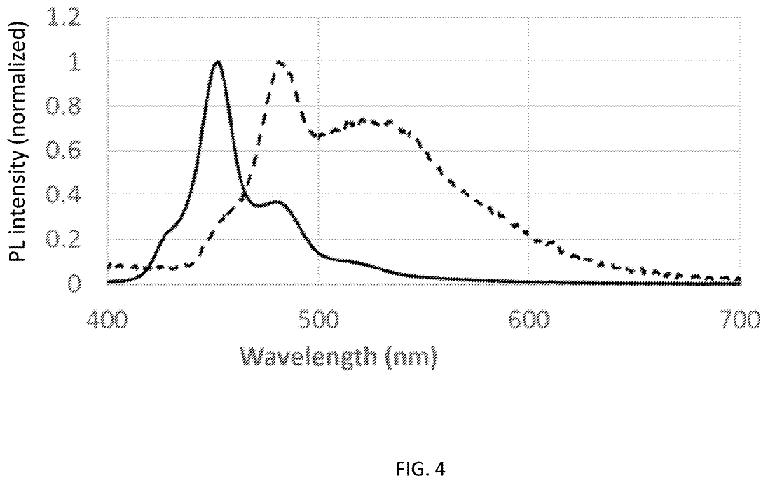

With reference to Table 1 and FIGS. 3 and 4, “uncaged” Example Compounds 1 and 3, i.e. photolysed Compounds 1 and 3, have red-shifted emission as compared to the “caged” Compound Examples 1 and 3. Uncaged Compound Example 1 has higher PLQY than its caged form.

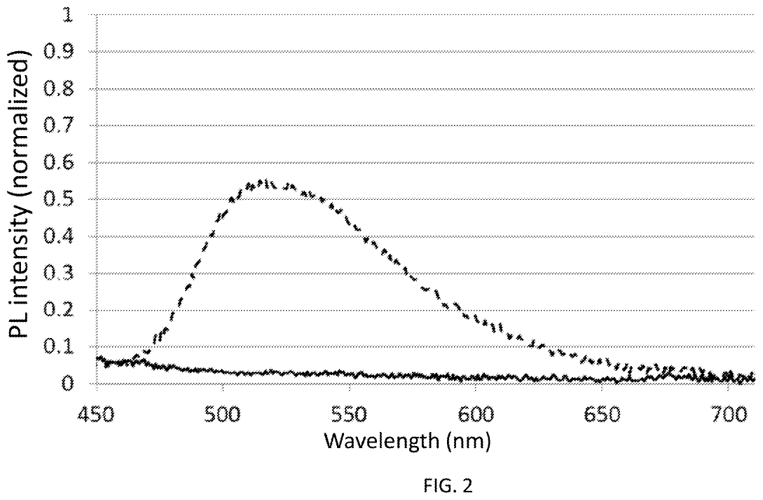

Emission spectra of caged and uncaged Comparative Compound 1, Compound Example 1 and Compound Example 3 are shown in FIGS. 2, 3 and 4 respectively in which caged compound emission is shown as a solid line and uncaged compound emission is shown as a dashed line.

TABLE 1

| Compound | PLQY | CIE coordinates |
|---|---|---|
| Comparative | 2% | (0.26, 0.24) |
| Compound 1 | | |
| Comparative | 27% | (0.31, 0.57) |
| Compound 1 uncaged | | |
| Example 1 | 15% | (0.18, 0.09) |
| Example 1 uncaged | 42% | (0.35, 0.60) |
| Example 3 | 51% | (0.15, 0.09) |
| Example 3 uncaged | 15% | (0.26, 0.46) |

2-Photon Cross-Section

A 2-photon cross-section of the compounds was modelled.

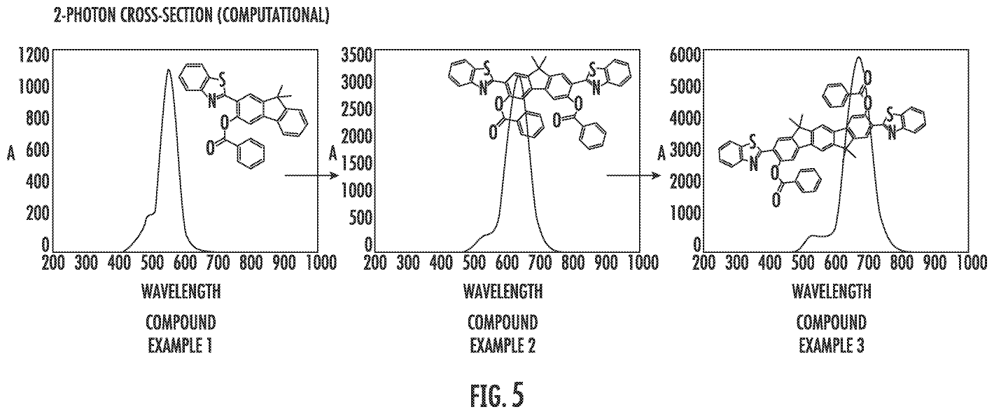

With reference to FIG. 5, the cross-section increases with degree of conjugation. Without wishing to be bound by any theory, the rigid 2-dimensional structure of the conjugated compounds enhances electron density in energy transitions.

Modelling was performed using TD-DFT methods, similar to those set out in J. Phys. Chem. C, 2013, 117, 18170-18189 using Gaussian16 software to calculate the following:
The ground state dipole moment
The transition state dipole moments (From TD-DFT)
The excited state dipole moments (From TD-DFT)
The transition dipole moments between excited states.

The “Sum over states” method was used to calculate the spectra. The rotationally averaged two-photon absorption strength was calculated at each transition, and a line-broadening factor was applied, as set out in J. Phys. Chem. C, 2013, 117, 18170-18189.

Data Writing

A solution of 1.8 g of PMMA, 0.2 g of Compound Example 2 and 14.9 g of chloroform was spin-coated onto a glass substrate at a speed of 1000 rpm to form a 6 micron thick film.

For 1 photon absorption a continuous 420 nm laser was used, as a power of 2 mW.

For 2 photon absorption a 532 nm pulsed laser was used emitting Ins pulses at a frequency of 20 Hz and a pulse power of 20 μW.

For both lasers the laser light was focused through a 5X objective with numerical aperture of 0.15 onto a sample moving at 1 μm/s.

For reading, an Olympus BX60 fluorescence microscope was used with a customised 400 nm LED illuminator and a 450 nm longpass filter. Fluorescence was recorded with a monochrome camera.

A region of the film was irradiated with a laser of wavelength of 420 nm moving in a continuous line across the substrate to achieve 1-photon absorption photolysis.

Another region of the film was irradiated with a laser of wavelength of 532 nm moving in a continuous line across the substrate to achieve 2-photon absorption photolysis.

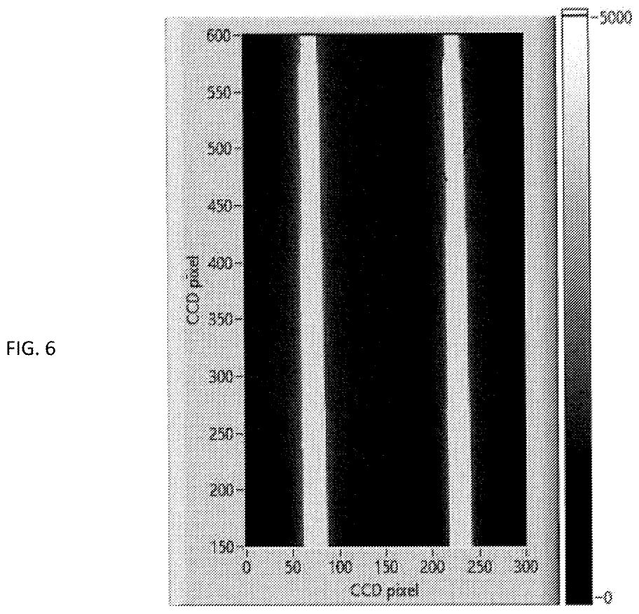

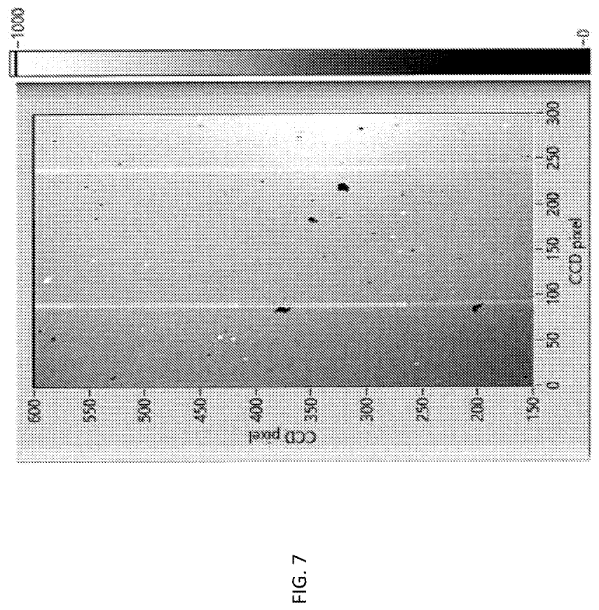

With reference to FIGS. 6 and 7, regions of higher luminance are observed in both of the irradiated regions, indicative of successful 1-photon and 2-photon writing.

What is claimed is:

1. A compound of formula (Ia), (Ib) or (Ic): 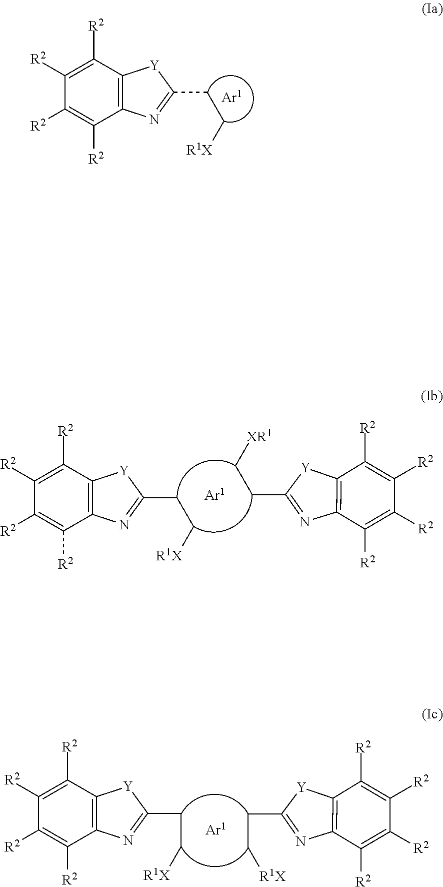
wherein:
Ar¹ is a fused aromatic or heteroaromatic group:
R² in each occurrence is H or a substituent;
Y is O, S or NR⁹ wherein R⁹ is H or a substituent;
and, for each group of formula (II), a ring carbon atom of Ar¹ adjacent to a ring carbon atom bound to the group of formula (II) is substituted with a group of formula —XR¹ wherein X is O, S or NR³ wherein R³ is H or C₁₋₁₂ alkyl and R¹ is a photocleavable group, wherein:
wherein Ar¹ is selected from formulae (IIIa) and (IIIb) for compounds of formula (Ia) and wherein Ar¹ is selected from formulae (IVa) and (IVb) for compounds of formula (Tb) or (Ic): 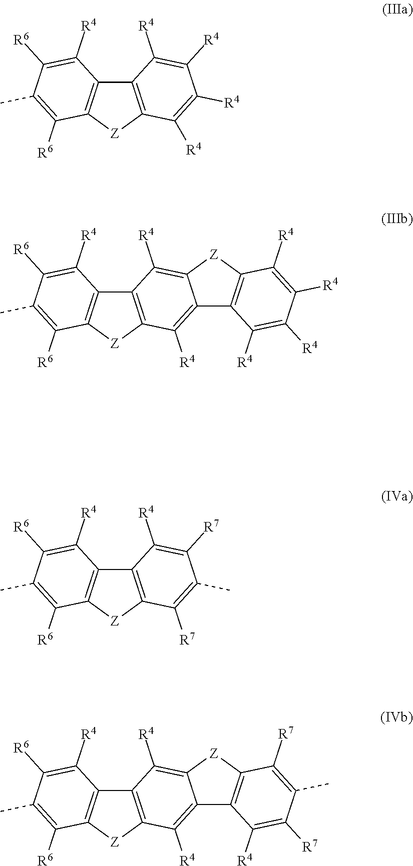
wherein:
Z is O, S, NR¹³, CR⁵₂ or SiR⁵₂ wherein R⁵ in each occurrence is independently a substituent and R¹³ is H or a substituent:
R⁴ in each occurrence is independently H or a substituent;
each R⁶ is H or a substituent with the proviso that one R⁶ is XR¹; and
each R⁷ is H or a substituent with the proviso that one R⁷ is XR¹.

2. The compound according to claim 1 wherein Ar¹ is a fused aromatic group.

3. The compound according to claim 1 wherein R¹ is a group of formula (V):
—C(═O)R⁸ (v)
wherein R⁸ is a substituent.

4. A composition comprising a compound according to claim 1 dispersed in a matrix.

5. A method of photolysis of a compound according to claim 1 comprising irradiating the compound with light of having a wavelength the same as or shorter than an absorption peak of the compound.

6. A method of photolysis of a compound according to claim 1 comprising irradiating the compound with light of having a wavelength longer than an absorption peak of the compound.

7. A recording medium comprising a layer comprising a compound according to claim 1.

8. A method of writing data to the recording medium according to claim 7 comprising exposing selected regions of the recording medium to a photolysing beam.
